(19) United States
(12) Patent Application Publication
(10) Pub. No.: US 2025/0135149 A1
(43) Pub. Date: May 1, 2025

(54) METHOD AND ARTIFICIAL RESUSCITATOR DEVICE FOR CONTROLLABLE VENTILATION VOLUME

(71) Applicant: GUANGZHOU LANDSWICK MEDICAL TECHNOLOGIES LTD., Guangzhou (CN)

(72) Inventors: Caixin SUN, Guangzhou (CN); Yueqi HUANG, Guangzhou (CN); Yuheng ZHAO, Guangzhou (CN); Hui DONG, Guangzhou (CN)

(21) Appl. No.: 18/751,363

(22) Filed: Jun. 24, 2024

Related U.S. Application Data
(63) Continuation of application No. PCT/CN2024/086841, filed on Apr. 9, 2024.

(30) Foreign Application Priority Data
Oct. 26, 2023 (CN) 202311400283.4

(51) Int. Cl. A61M 16/20; A61M 16/00

(52) U.S. Cl. CPC A61M 16/202; A61M 16/0084; A61M 16/209; A61M 2016/0027; A61M 2016/0033; A61M 2230/40

(57) ABSTRACT

A method and an artificial resuscitator device for controllable ventilation volume are provided. The artificial resuscitator includes: a gas control valve and a flow sensor set on an exhaust pipe of a respirator; a connecting tube branching off the exhaust pipe, with a three-way valve on a connecting tube that selectively communicates with a control side of the gas control valve, and a pressure relief opening on a side of the three-way valve that selectively communicates with the control side of the gas control valve; a control module that obtains a ventilation volume of the respirator based on a gas flow detected by the flow sensor and controls whether the three-way valve switches to a gas path according to the ventilation volume, to open or close the exhaust pipe through the gas control valve.

### TECHNICAL FIELD

The present invention relates to the field of medical devices, more specifically, to a method and an artificial resuscitator device for controllable ventilation volume.

### BACKGROUND

An artificial resuscitator, also known as a simple breathing apparatus, is primarily used to provide forced ventilation to patients without breathing and assist breathing-impaired patients. Compared to mouth-to-mouth respiration, the artificial resuscitator offers higher oxygen concentration and is easier to operate. In critical conditions where there is no time for intubation, a pressurized mask can be used to deliver oxygen directly, providing patients with sufficient oxygen supply to improve oxygen-deficient states.

The artificial resuscitator is characterized by their simple structure, quick and convenient operation, and portability. The artificial resuscitator mainly consists of an elastic breathing bag, a respirator, a breathing valve, a gas storage bag, a mask or a tracheal tube interface, and an oxygen interface. Oxygen enters a spherical bag and the gas storage bag, and by manually pressing the bag, oxygen is pushed through the front valve into a mask or tracheal tube closely attached to the patient's nose or mouth to achieve artificial ventilation.

However, manual operation of the artificial resuscitator requires the operator to manually press the bag for air supply, making it difficult to effectively control the ventilation volume. Even when operated by trained medical personnel, maintaining an appropriate ventilation volume for an extended period is challenging.

Therefore, there is a need to propose a method and an artificial resuscitator device for controllable ventilation volume to at least partially address the issues in the existing technology.

### SUMMARY

A series of simplified concepts are introduced in the summary of the present invention, which will be further elaborated in embodiments of the present invention. The summary of the present invention does not imply an attempt to limit key and necessary technical features of technical solutions described in the present invention, nor does it imply an attempt to determine the scope of the protection of the technical solutions described in the present invention.

To at least partially address the above problems, the present invention provides an artificial resuscitator device, including: a gas control valve and a flow sensor set on an exhaust pipe of a respirator; a connecting tube branching off the exhaust pipe, with a three-way valve on the connecting tube that selectively communicates with a control side of the gas control valve, and a pressure relief opening on a side of the three-way valve that selectively communicates with the control side of the gas control valve; a control module for obtaining a current ventilation volume of the respirator based on a gas flow detected by the flow sensor, and controlling whether the three-way valve switches to a gas path based on the current ventilation volume to open or close the exhaust pipe through the gas control valve.

Preferably, a basis for the control module to control whether the three-way valve switches to the gas path is to compare the obtained current ventilation volume of the respirator with a set ventilation volume. If the current ventilation volume of the respirator reaches the set ventilation volume, the control module switches the three-way valve to a locked gas path, thereby connecting the exhaust pipe to the connecting tube, causing the control side of the gas control valve to be pressurized and the exhaust pipe to be closed; and if the current ventilation volume of the respirator does not reach the set ventilation volume, the control module maintains a pressure relief gas path, thereby connecting the connecting tube to the pressure relief opening, with the control side of the gas control valve not under pressure, and keeping the exhaust pipe open.

Preferably, the respirator includes a gas storage bag with an inlet one-way valve disposed at an intake end of the gas storage bag and an outlet one-way valve disposed at an exhaust end of the gas storage bag and connected to the exhaust pipe.

Preferably, additionally, the artificial resuscitator device further includes a frequency dynamic indication module for prompting an operator to perform ventilation operations based on a set breathing time interval of a set frequency, while controlling the three-way valve to switch to a pressure relief gas path through the control module. After ventilation, when the ventilation volume of the respirator reaches the set ventilation volume and has not reached the next ventilation time node, the control module switches the three-way valve to a locked gas path.

Preferably, the control module and the frequency dynamic indication module are composed of circuits to realize corresponding function thereof.

Preferably, the exhaust pipe includes a first pipe section connected to the exhaust end of the gas storage bag and a second pipe section connected to a gas guiding section. The gas control valve is located at a junction of the first pipe section and the second pipe section, the connecting tube is branched off at the first pipe section, and the flow sensor is located on the second pipe section.

Preferably, an end of the first pipe section communicating with the second pipe section is located inside the second pipe section to form an annular connecting section, and the gas control valve is located at an end of the second pipe section near the annular connecting section.

The gas control valve includes a pressure chamber body connected between the second pipe section and the connecting tube, with the three-way valve located at a connection between the connecting tube and the pressure chamber body, and the pressure relief opening selectively communicating with the pressure chamber body through the three-way valve. A diaphragm assembly A is located at a connection between the second pipe section and the pressure chamber body, selectively sealing the end of the first pipe section inside the second pipe section. A side of the diaphragm assembly A facing towards the pressure chamber body is a control side of the diaphragm assembly A, a side of the diaphragm assembly A facing towards the annular connecting section is a ventilation side of the diaphragm assembly A, and an area under gas pressure of the control side of the diaphragm assembly A is greater than an area under gas pressure of the ventilation side of the diaphragm assembly A.

Preferably, the gas control valve uses a diaphragm valve, including a valve seat for connecting the first pipe section and the second pipe section, with a valve cavity inside the valve seat separated into two communicating cavities by a partition plate. A valve cover is located on a side of the valve seat, with a diaphragm assembly B inside the valve cover for selectively sealing the two communicating cavities connected to the first pipe section. A side of the valve cover facing away from the valve seat is connected to the connecting tube via a pressure tube, with the three-way valve located at a connection between the connecting tube and the pressure tube, and the pressure relief opening selectively communicating with the pressure tube through the three-way valve. A side of the diaphragm assembly B facing towards the pressure tube is a control side of the diaphragm assembly B and a side of the diaphragm assembly B facing towards the two communicating cavities is a ventilation side of the diaphragm assembly B.

Preferably, the diaphragm assembly A includes a diaphragm main body, with two ends of an outer ring area of the diaphragm main body connected to the second pipe section and the pressure chamber body by a first fixing ring and a second fixing ring.

Preferably, a side of the first fixing ring facing towards the diaphragm main body is uniformly arranged with multiple first grooves, and a side of the second fixing ring facing towards the diaphragm main body is uniformly arranged with multiple second grooves. One of the multiple first grooves contains a first pressure sensor in contact with the diaphragm main body, and one of the multiple second grooves contains a second pressure sensor in contact with the diaphragm main body.

The first pressure sensor and the second pressure sensor are both connected to the control module to sense force situation on the ventilation side of the diaphragm main body and the control side of the diaphragm assembly A to determine if the diaphragm main body is functioning properly.

A controllable ventilation volume method for the artificial resuscitator device includes the following steps: obtaining the set frequency and set ventilation volume based on the user's required breathing frequency and single ventilation volume; controlling the exhaust pipe of the respirator to be in the open state, obtaining the set breathing time interval based on the set frequency; real-time monitoring the gas flow at the exhaust pipe, obtaining the current ventilation volume of the respirator, and determining if the current ventilation volume meets the set ventilation volume.

If the current ventilation volume of the respirator does not meet the set ventilation volume, the exhaust pipe of the respirator keeps open; and if the current ventilation volume of the respirator meets the set ventilation volume, the exhaust pipe of the respirator closes and the ventilation time interval in real-time is monitored.

If the ventilation time interval does not reach the set breathing time interval of the set frequency, the exhaust pipe of the respirator keeps closed; and if the ventilation time interval reaches the set breathing time interval of the set frequency, the exhaust pipe of the respirator opens and the operator is prompted for the next ventilation operation.

Compared to the prior art, the present invention has the following beneficial effects:

The controllable ventilation volume method and artificial resuscitator device of the present invention use the three-way valve and the gas control valve to form a valve switch used for controlling the opening or closing of the exhaust pipe. Compared to a high-flow and high-power consumption valve switch, the present invention uses the low-power and small-aperture three-way valve. The control module can indirectly control the opening and closing of the gas control valve by controlling the three-way valve, reducing the power consumption of the artificial resuscitator device. It can effectively provide feedback on manual respiratory actions, cut off ventilation exceeding the required tidal volume, achieve quantitative ventilation, and retain the advantages of traditional artificial resuscitators while being compact and portable.

Other advantages, objectives, and features of the controllable ventilation volume method and artificial resuscitator device of the present invention will be partially illustrated in the following description and will be understood by those skilled in the art through research and practice of the invention in this field.

### BRIEF DESCRIPTION OF THE DRAWINGS

The drawings are used to provide a further understanding of the present invention and constitute a part of the specification. They are used in conjunction with the embodiments of the present invention to explain the invention and do not limit the invention. In the drawings:

FIG. 1 is a schematic diagram of the control of the artificial resuscitator device of the present invention;

FIG. 2 is a schematic diagram of the gas path principle of the artificial resuscitator device of the present invention;

FIG. 3 is a schematic diagram of the first structure of the gas control valve when the three-way valve in the artificial resuscitator device of the present invention switches to the pressure relief gas path;

FIG. 4 is a schematic diagram of the first structure of the gas control valve when the three-way valve in the artificial resuscitator device of the present invention switches to the locked gas path;

FIG. 5 is a schematic diagram of the pressure on the ventilation side and control side of diaphragm assembly A in the artificial resuscitator device of the present invention when the three-way valve switches to the locked gas path;

FIG. 6 is a schematic diagram of the second structure of the gas control valve in the artificial resuscitator device of the present invention;

FIG. 7 is a schematic diagram of the structure of diaphragm assembly A in the artificial resuscitator device of the present invention;

FIG. 8 is a flowchart of the ventilation method with controllable ventilation volume of the artificial resuscitator device of the present invention.

### DETAILED DESCRIPTION OF EMBODIMENTS

Further detailed description of the present invention will be provided below in conjunction with the drawings and embodiments to enable those skilled in the art to implement the invention based on the description.

It should be understood that terms such as “comprising,” “including,” and “having” used in this document do not exclude the presence or addition of one or more other elements or combinations thereof.

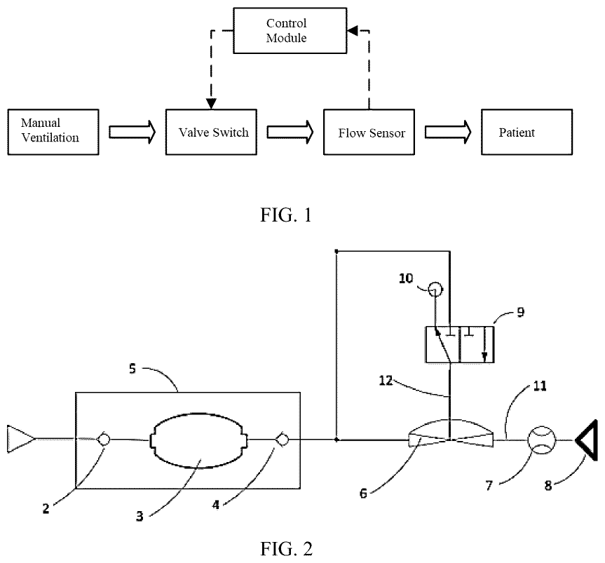

As shown in FIGS. 1 and 2, the present invention provides an artificial resuscitator device including: a gas control valve 6 and a flow sensor 7 set on an exhaust pipe 11 of a respirator 5; a connecting tube 12 is connected to the exhaust pipe 11, and a three-way valve 9 is provided on the connecting tube 12 to selectively communicate with a control side of the gas control valve 6, with a pressure relief opening 10 selectively communicating with the control side of the gas control valve 6 on a side of the three-way valve 9; a control module is used for obtaining the current ventilation volume of the respirator 5 based on the gas flow detected by the flow sensor 7 and controlling whether the three-way valve 9 switches to the gas path based on the current ventilation volume to open or close the exhaust pipe 11 through the gas control valve 6.

Here, the ventilation volume refers to the single ventilation volume of the respirator 5, and the tidal volume refers to the amount of gas inhaled or exhaled during quiet breathing, which is related to age, gender, body surface area, breathing habits, and body metabolism; and the set ventilation volume should match the patient's tidal volume.

When manually operating the respirator 5, the three-way valve 9 switches to the pressure relief gas path to relieve pressure on the control side of the gas control valve 6, thus opening the exhaust pipe 11. The flow sensor 7 can detect the gas flow each time the respirator 5 ventilates the patient, and the control module can receive real-time flow signals detected by the flow sensor 7 to obtain the current ventilation volume of the respirator 5. When the ventilation volume meets the patient's required tidal volume, the three-way valve 9 switches to the locked gas path to pressurize the control side of the control valve 6, closing the exhaust pipe to provide a quantified ventilation volume each time.

The gas control valve 6 and the three-way valve 9 form the valve switch for controlling the opening or closing of the exhaust pipe 11. Traditional valve switches require high power consumption to ensure a large ventilation flow, which does not meet the portability requirements. In contrast, the present invention uses low-power and small-aperture three-way valves. By controlling the three-way valve 9, the control module can indirectly control the opening or closing of the gas control valve 6, reducing power consumption of the artificial resuscitator device. It can effectively provide feedback on manual respiratory actions, cut off ventilation volumes higher than the required tidal volume to achieve quantitative ventilation, and the artificial resuscitator device is compact and portable, retaining the advantages of traditional artificial resuscitators.

In an embodiment, the control module controls whether the three-way valve 9 switches to the gas path based on comparing the current ventilation volume of the respirator 5 with the set ventilation volume. If the current ventilation volume of the respirator 5 reaches the set ventilation volume, the three-way valve 9 switches to the locked gas path, connecting the exhaust pipe 11 to the connecting tube 12, pressurizing the control side of the gas control valve 6 to close the exhaust pipe 11. If the current ventilation volume of the respirator 5 does not reach the set ventilation volume, the three-way valve 9 maintains the current pressure relief gas path, connecting the connecting tube 12 to the pressure relief opening 10, and the control side of the gas control valve 6 is not pressurized, keeping the exhaust pipe 11 open.

The set ventilation volume is the tidal volume required by the patient. By monitoring the gas flow at the exhaust pipe 11 in real-time using the flow sensor 7, the current ventilation volume of the respirator 5 is obtained and compared with the set ventilation volume to control the switching of the three-way valve 9, ensuring that the single ventilation volume meets the patient's tidal volume requirements and preventing excessive ventilation that could harm the patient, achieving the function of quantitative ventilation.

In an embodiment, the respirator 5 includes a gas storage bag 3, with an inlet one-way valve 2 at its intake end and an outlet one-way valve 4 at its exhaust end connected to the exhaust pipe 11.

The gas storage bag 3 can be pressed by the operator to provide the required oxygen or air to the patient. The inlet one-way valve 2 allows gas to enter the gas storage bag 3 only from the intake end, while the outlet one-way valve 4 allows gas from the gas storage bag 3 to be discharged only to the exhaust pipe 11.

In an embodiment, the artificial resuscitator device further includes a frequency dynamic indication module for prompting the operator to perform ventilation operations based on the set breathing time interval of the set frequency and controlling the three-way valve 9 to switch to the pressure relief gas path; after ventilation, i.e., when the ventilation volume of the respirator 5 reaches the set ventilation volume and has not reached the next ventilation time node, the control module controls the three-way valve 9 to switch to the locked gas path.

To provide a more accurate ventilation frequency for the patient, the ventilation frequency of the artificial resuscitator device is set based on the patient's respiratory frequency requirements, i.e., the set frequency. Specifically, based on the inhalation time interval of the set frequency, the operator is prompted to perform ventilation operations (pressing the gas storage bag 3). For example, if the respiratory frequency per minute is set to 20 breaths per minute (set frequency) and each breath takes 3 seconds, the set breathing time interval is 3 seconds, with a prompt every 3 seconds for the operator to press the gas storage bag 3. The prompt can be displayed on the screen or through audio-visual alerts.

Furthermore, the frequency dynamic indication module is electrically connected to the control module. When the current ventilation volume of the respirator 5 reaches the set ventilation volume and has not reached the next ventilation time node (the ventilation time interval has not reached the preset ventilation time interval, no ventilation operation is prompted), the three-way valve 9 remains in the locked gas path to prevent accidental over-pressing of the gas storage bag 3 and excessive ventilation to the patient, ensuring safety.

In an embodiment, the exhaust pipe 11 includes a first pipe section 111 connected to the exhaust end of the gas storage bag 3 and a second pipe section 112 connected to a gas guiding section 8; the gas control valve 6 is set at the connection between the first pipe section 111 and the second pipe section 112, the connecting tube 12 bypasses the first pipe section 111, and the flow sensor 7 is set on the second pipe section 112.

The gas guiding section 8 is a mask or tracheal tube used for patient ventilation.

Furthermore, two structural forms of the gas control valve 6 are provided as follows.

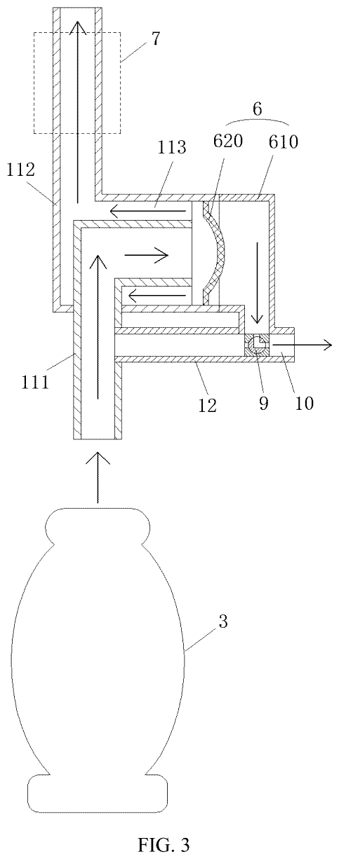

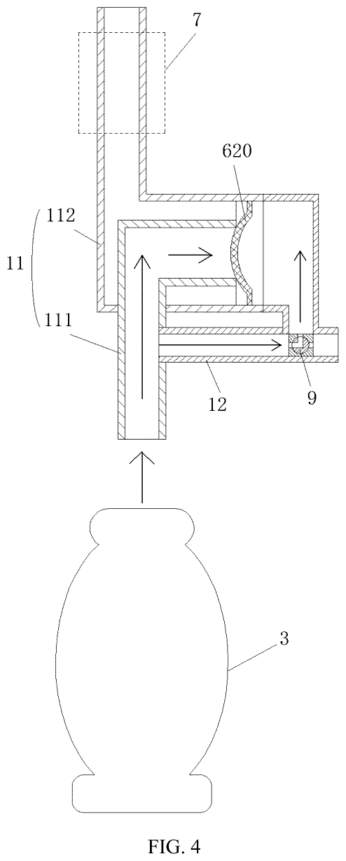

As shown in FIGS. 3 and 4, the first structure of the gas control valve 6 is provided, an end of the first pipe section 111 communicating with the second pipe section 112 is located inside the second pipe section 112, forming an annular connecting section 113, and the gas control valve 6 is set at an end of the second pipe section 112 close to the annular connecting section 113.

The gas control valve 6 includes a pressure chamber body 610 connected to the second pipe section 112 at an end and to the connecting tube 12 at the other end, the three-way valve 9 is set at the connection between the connecting tube 12 and the pressure chamber body 610, and the pressure relief opening 10 selectively communicates with the pressure chamber body 610 through the three-way valve 9; a diaphragm assembly A 620 is set at the connection between the second pipe section 112 and the pressure chamber body 610, the diaphragm assembly A 620 can selectively seal the end of the first pipe section 111 inside the second pipe section 112; a side of the diaphragm assembly A 620 near the pressure chamber body 610 is the control side of the diaphragm assembly A 620, and the other side near the annular connecting section 113 is the ventilation side of the diaphragm assembly A 620, with the control side of the diaphragm assembly A 620 having a larger area subjected to gas pressure than the ventilation side of the diaphragm assembly A 620.

As shown in FIG. 3, when ventilation is required for the patient, the control module controls the three-way valve 9 to switch to the pressure relief gas path, disconnecting the connecting tube 12 from the control side of the gas control valve 6, connecting the pressure relief opening 10 to the control side of the gas control valve 6, allowing the gas in the pressure chamber body 610 to be released through the pressure relief opening 10, releasing the pressure. The gas discharged from the first pipe section 111 acts on the diaphragm of the diaphragm assembly A 620, causing the ventilation side of the diaphragm assembly A 620 to be subjected to greater force than the control side, deforming the diaphragm of the diaphragm assembly A 620 towards the control side, connecting the end of the first pipe section 111 to the annular connecting section 113, opening the valve switch, thus keeping the exhaust pipe 11 open;

As shown in FIG. 4, when ventilation is not required for the patient, the control module controls the three-way valve 9 to switch to the locked gas path, connecting the connecting tube 12 to the control side of the gas control valve 6, connecting the first pipe section 111 to the pressure chamber body 610, allowing the gas paths of the ventilation side and control side of the diaphragm assembly A 620 to be connected, with the pressure on both sides of the diaphragm assembly A 620 being consistent. Since the diaphragm assembly A 620 is designed with a larger pressure area on the control side than on the ventilation side, the control side experiences greater force than the ventilation side. At this point, the diaphragm of the diaphragm assembly A 620 deforms towards the ventilation side, sealing the end of the first pipe section 111, disconnecting the first pipe section 111 from the annular connecting section 113, closing the valve switch, and thus closing the exhaust pipe 11.

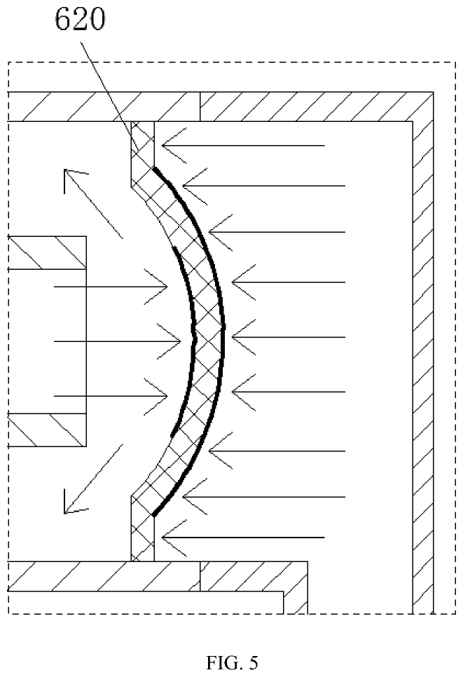

As shown in FIG. 5, when the three-way valve 9 switches to the locked gas path, the schematic diagram shows the pressure on the ventilation side and control side of the diaphragm assembly A 620, with the thick black line on the right indicating the pressure on the control side and the thick black line on the left indicating the pressure on the ventilation side, where the pressure area on the ventilation side is smaller than that on the control side.

Through the above structure, the use of low-power and small-aperture three-way valve 9 in conjunction with the diaphragm assembly A 620 can achieve the opening or closing of the valve switch. The diaphragm assembly A 620 does not require electric control; by changing the gas path through the three-way valve 9, the forces on the ventilation side and control side of the diaphragm assembly A 620 can be altered to achieve the function of the valve switch. Compared to using the high-power valve switch, the electric control power consumption is reduced, quantitative ventilation is achieved, and the portability of the artificial resuscitator is maintained.

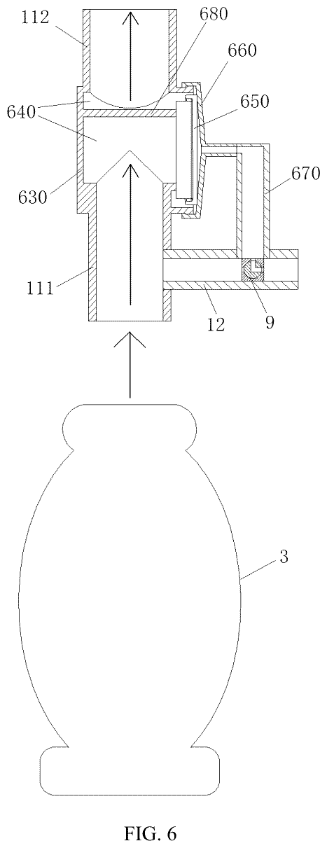

As shown in FIG. 6, a second structure of the gas control valve 6 is illustrated. The gas control valve 6 adopts a diaphragm valve, including: a valve seat 630 for connecting the first pipe section 111 and the second pipe section 112; the valve seat 630 defines a valve cavity therein, and a partition plate 680 inside the valve cavity divides the valve cavity into two communicating cavities 640. Positioned on a side of the valve seat 630 is a valve cover 660, inside of which a diaphragm assembly B 650 is located. The diaphragm assembly B 650 selectively seals the communicating cavity 640 that communicates with the first pipe section 111. The valve cover 660, on the side away from the valve seat 630, communicates with the connecting tube 12 via a pressure tube 670, with the three-way valve 9 being positioned at the connection between the connecting tube 12 and the pressure tube 670. The pressure relief opening 10 selectively communicates with the pressure tube 670 through the three-way valve 9. A side of the diaphragm assembly B 650 facing towards the pressure tube 670 is a control side of the diaphragm assembly B 650 and a side of the diaphragm assembly B 650 facing towards the two communicating cavities 640 is a ventilation side of the diaphragm assembly B 650.

The valve cover 660 seals one side of the valve chamber, with the diaphragm assembly B 650 sealingly connected to the valve cover 660. The diaphragm assembly B 650, away from the communicating cavity 640, forms the control side with the valve cover 660, while the side near the communicating cavity 640 serves as the ventilation side.

When ventilation to the patient is required, the control module switches the three-way valve 9 to the pressure relief gas path, disconnecting the connecting tube 12 from the pressure tube 670. This action allows the pressure tube 670 to communicate with the pressure relief opening 10, enabling the control side to release pressure. Gas from the first pipe section 111 enters the communicating cavity 640 connected thereto, exerting pressure on the ventilation side of the diaphragm assembly B 650, causing the diaphragm of the diaphragm assembly B 650 to deform towards the control side, thereby connecting the two communicating cavities 640 and opening the switching valve, with the exhaust pipe 11 in the open state.

When ventilation to the patient is not required, the control module switches the three-way valve 9 to the locked gas path. The pressure tube 670 is not connected to the pressure relief opening 10, and the connecting tube 12 is connected to the pressure tube 670. This connection allows the communicating cavity 640 connected to the first pipe section 111 to communicate with the pressure tube 670. The pressure on the ventilation side and the control side of the diaphragm assembly B 650 is equal. When the diaphragm assembly B 650 is set with the same pressure on the control side and the ventilation side thereof (similar to the principle of the diaphragm assembly A 620) or when the pressure exerted on the control side of the diaphragm assembly B 650 is greater than that exerted on the ventilation side of the diaphragm assembly B 650, the diaphragm of the diaphragm assembly B 650 will deform and move towards the ventilation side thereof, sealing the communicating cavity 640, making the first pipe section 111 and the second pipe section 112 disconnected, thereby closing the switching valve to make the exhaust pipe 11 in a close state.

To ensure the sealing of the diaphragm assembly B 650 to the communicating cavity 640, an elastic member that assists the diaphragm assembly B 650 in moving towards the ventilation side can be selectively placed on the control side. The combined force of the gas pressure and the elastic member on the control side is greater than the gas pressure on the ventilation side, thereby sealing the communicating cavity 640. The elastic force of the elastic member is small and does not cause the diaphragm of the diaphragm assembly B 650 to deform and move when acting alone.

Through the above structure, a low-power and small-aperture three-way valve 9 can be used in conjunction with the diaphragm assembly A 620 to achieve the opening or closing of the switching valve, enabling quantitative ventilation while retaining the portability of the artificial resuscitator device.

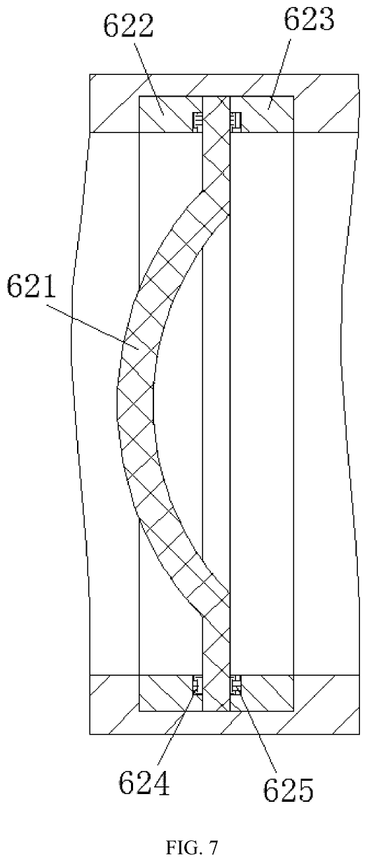

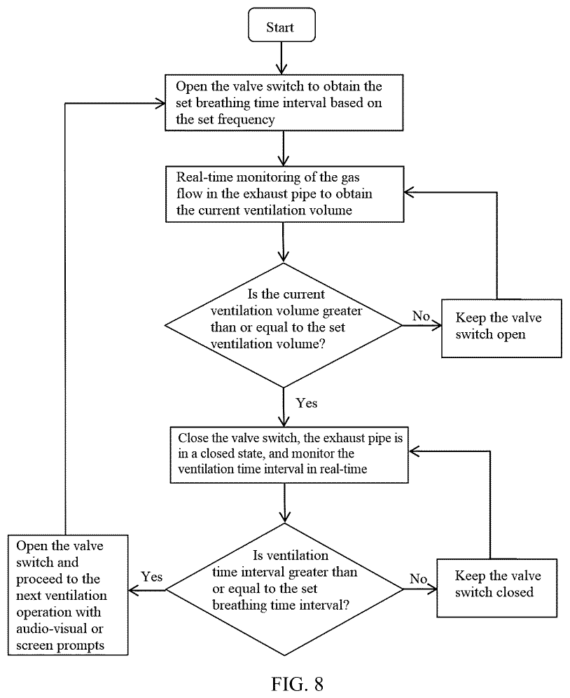

As shown in FIG. 7, in an embodiment, the diaphragm assembly A 620 includes a diaphragm main body 621, with its outer ring area connected to the second pipe section 112 and the pressure chamber body 610 by the first fixing ring 622 and the second fixing ring 623.

The outer ring area of the diaphragm main body 621 is clamped between the first fixing ring 622 and the second fixing ring 623, which are sealingly connected to the second pipe section 112 or the pressure chamber body 610, thereby securing the diaphragm main body 621.

Additionally, the structure of the diaphragm assembly B 650 can be configured to include an installation shell that is sealingly connected to the valve cover 660, with passages on both sides of the installation shell. The diaphragm main body 621 is connected to the installation shell through the first fixing ring 622 and the second fixing ring 623 on its outer ring area.

Furthermore, the first fixing ring 622 is uniformly provided with multiple first grooves on a side near the diaphragm main body 621, and the second fixing ring 623 is uniformly provided with multiple second grooves on a side near the diaphragm main body 621. One of the multiple first grooves is provided with a first pressure sensor 624 in contact with the diaphragm main body 621, while one of the multiple second grooves is provided with a second pressure sensor 625 in contact with the diaphragm main body 621.

The first pressure sensor 624 and the second pressure sensor 625 are both connected to the control module to sense the force on the ventilation side and the control side of the diaphragm assembly A 620, determining the normal operation of the diaphragm main body 621.

The first pressure sensor 624 is positioned on the ventilation side of the diaphragm assembly A 620, and the second pressure sensor 625 is positioned on the control side of the diaphragm assembly A 620. If the force on the ventilation side of the diaphragm assembly A 620 is greater than that on the control side of the diaphragm assembly A 620, causing the diaphragm main body 621 to move and deform towards the control side of the diaphragm assembly A 620, the second pressure sensor 625 experiences compression, resulting in a change in the pressure value detected by the second pressure sensor 625. If the force on the control side of the diaphragm assembly A 620 is greater than that on the ventilation side of the diaphragm assembly A 620, causing the diaphragm main body 621 to move and deform towards the ventilation side of the diaphragm assembly A 620, the first pressure sensor 624 experiences compression, leading to a change in the pressure value detected by the first pressure sensor 624.

During normal operation of the switching valve, when the diaphragm main body 621 moves and deforms, the maximum pressure values detected by the first pressure sensor 624 and the second pressure sensor 625 (the maximum gas pressure generated on the control side or the ventilation side when manually pressing the gas storage bag 3) should exceed the preset pressure value. The preset pressure value is the pressure required to deform the diaphragm main body 621.

The movement and deformation of the diaphragm main body 621 occur when switching the three-way valve 9 gas paths. Therefore, each time the three-way valve 9 gas paths are switched, the control module obtains the maximum pressure values detected by the first pressure sensor 624 and the second pressure sensor 625, compares them with the preset pressure value, and if the maximum pressure value is less than the preset pressure value, an alarm is issued. This indicates damage to the diaphragm main body 621 or leakage on at least one side of the ventilation side and the control side, requiring cessation of use to prevent inappropriate ventilation and potential harm to the patient. In critical situations, the ventilation volume is displayed on the screen of the manual resuscitator device. If the diaphragm main body 621 is working abnormally but still allows ventilation, the observed ventilation volume can be used to manually control the pressing and stopping of ventilation by the gas storage bag 3.

Prior to each use of this manual resuscitator device, a test can be conducted (without the gas guiding section 8 in contact with the patient) by manually pressing the gas storage bag 3 to check for any alarm prompts. If an alarm is triggered, it indicates that the artificial resuscitator device is malfunctioning; if no alarm is triggered, the device can be used normally.

A method for controllable ventilation volume in the artificial resuscitator device includes the following steps.

A set frequency and a set ventilation volume are obtained based on the user's required breathing frequency and single ventilation volume.

The exhaust pipe 11 of the respirator 5 is controlled to be in the open state and a set breathing time interval of a set frequency is obtained based on the set frequency.

Real-time gas flow at the exhaust pipe 11 and the current ventilation volume of the respirator 5 are obtained to determine whether the current ventilation volume of the respirator 5 meets the set ventilation volume.

If the current ventilation volume of the respirator 5 does not meet the set ventilation volume, the exhaust pipe 11 of the respirator 5 is kept open; and if the current ventilation volume of the respirator 5 meets the set ventilation volume, the exhaust pipe 11 of the respirator 5 is closed and the ventilation time interval frequency is monitored in real time.

If the ventilation time interval does not reach the set breathing time interval of the set frequency, the exhaust pipe 11 of the respirator 5 is kept closed; and if the ventilation time interval reaches the set breathing time interval of the set frequency, the exhaust pipe 11 of the respirator 5 is open and the operator is prompted to perform the next ventilation operation.

By utilizing the above method, frequency control is assisted, operators are reminded to perform ventilation operations, ensuring that the ventilation frequency to the patient aligns with their breathing frequency. The exhaust pipe 11 is controlled to open based on the set frequency time intervals, remaining closed at other unsuitable times to prevent improper ventilation by the respirator 5 and ensure safety during use.

Real-time monitoring of the gas flow at the exhaust pipe 11, integrating over time, allows for obtaining the single ventilation volume of the respirator 5. When the set ventilation volume is reached, ventilation is stopped by closing the exhaust pipe 11, achieving quantitative ventilation while preventing over-ventilation and ensuring the safety of patient ventilation.

In the description of the present invention, it should be understood that terms such as “center,” “longitudinal,” “lateral,” “length,” “width,” “thickness,” “top,” “bottom,” “front,” “back,” “left,” “right,” “vertical,” “horizontal,” “top,” “bottom,” “inner,” “outer,” “clockwise,” “counterclockwise,” “axial,” “radial,” “circumferential,” and the like indicating orientation or positional relationships are based on the orientation or positional relationships shown in the drawings. These terms are used for ease of description and simplification, not to indicate or imply that the devices or components referred to must have specific orientations, be constructed or operated in specific orientations, and therefore should not be construed as limiting the present invention.

In the present invention, unless otherwise explicitly specified and limited, terms such as “mounting,” “connecting,” “linking,” “fixing,” and the like should be broadly interpreted. For example, it can be a fixed connection or a detachable connection, or integral; it can be a mechanical connection, an electrical connection, or communication with each other; it can be directly connected, or indirectly connected through an intermediate medium; it can be an internal connection of two components or an interaction relationship between two components, unless otherwise explicitly limited. For those skilled in the art, the specific meanings of the above terms in the present invention can be understood according to specific circumstances.

Although embodiments of the present invention are disclosed as above, it is not limited to the applications set forth in the specification and embodiments, it is fully applicable to various fields suitable for the present invention, and additional modifications can be readily accomplished by those familiar with the field, and therefore without departing from the general concepts defined by the claims and equivalents, the present invention is not limited to the particular details with which the illustrations are shown and described herein. The present invention is therefore not limited to the specific details and illustrations shown and described herein.

What is claimed is:

1. An artificial resuscitator device, comprising: a gas control valve (6) and a flow sensor (7) set on an exhaust pipe (11) of a respirator (5); a connecting tube (12) branching off the exhaust pipe (11), with a three-way valve (9) on the connecting tube (12) that selectively communicates with a control side of the gas control valve (6), and a side of the three-way valve (9) defines a pressure relief opening (10), and the pressure relief opening (10) selectively communicates with the control side of the gas control valve (6); a control module configured to obtain a current ventilation volume of the respirator (5) based on a gas flow detected by the flow sensor (7) and control whether the three-way valve (9) switches to a gas path according to the current ventilation volume, to open or close the exhaust pipe (11) through the gas control valve (6);
wherein the respirator (5) comprises a gas storage bag (3) with an inlet one-way valve (2) disposed at an intake end of the gas storage bag (3) and an outlet one-way valve (4) disposed at an exhaust end of the gas storage bag (3) and connected to the exhaust pipe (11); and
wherein the exhaust pipe (11) comprises a first pipe section (111) connected to the exhaust end of the gas storage bag (3) and a second pipe section (112) connected to a gas guiding section (8); the gas control valve (6) is set at a junction of the first pipe section (111) and the second pipe section (112), the connecting tube (12) is branched off at the first pipe section (111), and the flow sensor (7) is set on the second pipe section (112).

2. The artificial resuscitator device according to claim 1, wherein a basis for the control module to control whether the three-way valve (9) switches to the gas path comprises: comparing the current ventilation volume of the respirator (5) with a set ventilation volume; if the current ventilation volume of the respirator (5) reaches the set ventilation volume, controlling the control module to switch the three-way valve (9) to a locked gas path, connecting the exhaust pipe (11) to the connecting tube (12), causing the gas control valve (6) to close the exhaust pipe (11); if the current ventilation volume of the respirator (5) does not reach the set ventilation volume, controlling the control module to maintain a pressure relief gas path, connecting the connecting tube (12) to the pressure relief opening (10), and controlling the control side of the gas control valve (6) to remain unforced, keeping the exhaust pipe (11) open.

3. The artificial resuscitator device according to claim 1, further comprising: a frequency dynamic indication module, wherein the frequency dynamic indication module is configured to prompt an operator to perform ventilation operations based on a set breathing time interval of a set frequency, and control the three-way valve (9) to switch to a pressure relief gas path through the control module; after ventilation ends, when the current ventilation volume of the respirator (5) reaches a set ventilation volume and has not reached a next ventilation time node, the control module switches the three-way valve (9) to a locked gas path.

4. The artificial resuscitator device according to claim 1, wherein an end of the first pipe section (111) communicating with the second pipe section (112) is located inside the second pipe section (112) to form an annular connecting section (113), and the gas control valve (6) is set at an end of the second pipe section (112) near the annular connecting section (113); and
wherein the gas control valve (6) comprises a pressure chamber body (610) connected between the second pipe section (112) and the connecting tube (12), the three-way valve (9) is set at a connection between the connecting tube (12) and the pressure chamber body (610), and the pressure relief opening (10) selectively communicates with the pressure chamber body (610) through the three-way valve (9); a diaphragm assembly A (620) is set at a connection between the second pipe section (112) and the pressure chamber body (610), and the diaphragm assembly A (620) is capable of selectively sealing the end of the first pipe section (111) located inside the second pipe section (112); a side of the diaphragm assembly A (620) facing towards the pressure chamber body (610) is a control side of the diaphragm assembly A (620), a side of the diaphragm assembly A (620) facing towards the annular connecting section (113) is a ventilation side of the diaphragm assembly A (620), and an area under gas pressure of the control side of the diaphragm assembly A (620) is greater than an area under gas pressure of the ventilation side of the diaphragm assembly A (620).

5. The artificial resuscitator device according to claim 1, wherein the gas control valve (6) comprises a valve seat (630) configured to connect the first pipe section (111) and the second pipe section (112), the valve seat (630) defines a valve cavity therein, the valve seat (630) is provided with a partition plate (680) therein that is configured to separate the valve cavity into two communicating cavities (640); a valve cover (660) is set on a side of the valve seat (630), the valve cover (660) is provided with a diaphragm assembly B (650) therein, and the diaphragm assembly B (650) is configured to selectively seal the communicating cavity (640) connected to the first pipe section (111); a side of the valve cover (660) facing away from the valve seat (630) communicates with the connecting tube (12) through a pressure tube (670), the three-way valve (9) is set at a connection between the connecting tube (12) and the pressure tube (670), and the pressure relief opening (10) selectively communicates with the pressure tube (670) through the three-way valve (9); a side of the diaphragm assembly B (650) facing towards the pressure tube (670) is a control side of the diaphragm assembly B (650), a side of the diaphragm assembly B (650) facing towards the two communicating cavities (640) is a ventilation side of the diaphragm assembly B (650), and an area under gas pressure of the control side of the diaphragm assembly B (650) is greater than an area under gas pressure of the ventilation side of the diaphragm assembly B (650).

6. The artificial resuscitator device according to claim 4, wherein the diaphragm assembly A (620) comprises a diaphragm main body (621), and two ends of an outer ring area of the diaphragm main body (621) are connected to the second pipe section (112) and the pressure chamber body (610) by a first fixing ring (622) and a second fixing ring (623).

7. The artificial resuscitator device according to claim 6, wherein a side of the first fixing ring (622) facing towards the diaphragm main body (621) is uniformly arranged with a plurality of first grooves, and a side of the second fixing ring (623) facing towards the diaphragm main body (621) is uniformly arranged with a plurality of second grooves; one of the plurality of first grooves is equipped with a first pressure sensor (624) in contact with the diaphragm main body (621), and one of the plurality of second grooves is equipped with a second pressure sensor (625) in contact with the diaphragm main body (621); and
wherein the first pressure sensor (624) and the second pressure sensor (625) are connected to the control module to sense force situation on the ventilation side of the diaphragm assembly A (620) and the control side of the diaphragm assembly A (620), thereby to determine whether the diaphragm main body (621) is functioning properly.

8. An artificial resuscitator device, comprising:
a respirator (5), wherein the respirator (5) comprises an exhaust pipe (11) disposed on an end of the respirator (5);
a gas control valve (6) and a flow sensor (7), disposed on the exhaust pipe (11) of the respirator (5);
a gas guiding section (8), disposed on a side of the flow sensor (7) facing away from the gas control valve (6);
a three-way valve (9), connected with the gas control valve (6) and
a control module, configured to obtain a current ventilation volume of the respirator (5) based on a gas flow detected by the flow sensor (7) and control whether the three-way valve (9) switches, according to the current ventilation volume, to open or close the exhaust pipe (11) through the gas control valve (6).

9. The artificial resuscitator device according to claim 8, wherein the artificial resuscitator device further comprises: a connecting tube (12), the connecting tube (12) is provided with the three-way valve (9) thereon, and the three-way valve (9) is configured to selectively communicate with a control side of the gas control valve (6).

10. The artificial resuscitator device according to claim 9, wherein the artificial resuscitator device defines a pressure relief opening (10) disposed on a side of the three-way valve (9), and the pressure relief opening (10) is configured to selectively communicate with the control side of the gas control valve (6).

11. The artificial resuscitator device according to claim 8, wherein the respirator (5) further comprises: a gas storage bag (3), an inlet one-way valve (2), and an outlet one-way valve (4); and
wherein the inlet one-way valve (2) is disposed at an intake end of the gas storage bag (3) facing away from the gas control valve (6); and the outlet one-way valve (4) is disposed at an exhaust end of the gas storage bag (3) facing towards the gas control valve (6) and is connected to the exhaust pipe (11).

12. The artificial resuscitator device according to claim 11, wherein the exhaust pipe (11) comprises: a first pipe section (111) and a second pipe section (112);
wherein the first pipe section (111) is connected to the outlet one-way valve (4) on the exhaust end of the gas storage bag (3); the second pipe section (112) is connected to the gas guiding section (8) of the artificial resuscitator device; and an annular connecting section (113) is formed in an end of the first pipe section (111) communicating with the second pipe section (112) and is disposed inside the second pipe section (112); and
wherein the gas control valve (6) is disposed in an end of the second pipe section (112) facing towards the annular connecting section (113); and the flow sensor (7) is disposed on the second pipe section (112).

13. The artificial resuscitator device according to claim 12, wherein the gas control valve (6) comprises: a pressure chamber body (610) and a diaphragm assembly A (620);
wherein an end of the pressure chamber body (610) is communicated with the second pipe section (112), and another end of the pressure chamber body (610) is communicated with a connecting tube (12) of the artificial resuscitator device; the three-way valve (9) is disposed at a junction between the connecting tube (12) and the pressure chamber body (610); and a pressure relief opening (10) defined on the connecting tube (12) is configured to selectively communicate with the pressure chamber body (610) through the three-way valve (9);
wherein the diaphragm assembly A (620) is disposed at a junction between the second pipe section (112) and the pressure chamber body (610), and the diaphragm assembly A (620) is configured to selectively seal the end of the first pipe section (111) disposed inside the second pipe section (112); and
wherein a side of the diaphragm assembly A (620) facing towards the pressure chamber body (610) is a control side of the diaphragm assembly A (620), a side of the diaphragm assembly A (620) facing towards the annular connecting section (113) is a ventilation side of the diaphragm assembly A (620), and an area subjected to gas pressure of the control side of the diaphragm assembly A (620) is greater than an area subjected to gas pressure of the ventilation side of the diaphragm assembly A (620).

14. The artificial resuscitator device according to claim 13, wherein the diaphragm assembly A (620) comprises: a diaphragm main body (621), a first fixing ring (622), and a second fixing ring (623); and
wherein two ends of an outer ring area of the diaphragm main body (621) are connected to the second pipe section (112) and the pressure chamber body (610) respectively by the first fixing ring (622) and the second fixing ring (623).

15. The artificial resuscitator device according to claim 14, wherein a side of the first fixing ring (622) facing towards the diaphragm main body (621) defines a plurality of first grooves, and a side of the second fixing ring (623) facing towards the diaphragm main body (621) defines a plurality of second grooves;
wherein one of the plurality of first grooves is provided with a first pressure sensor (624) therein and the first pressure sensor (624) is in contact with the diaphragm main body (621); and one of the plurality of second grooves is provided with a second pressure sensor (625) therein and the second pressure sensor (625) is in contact with the diaphragm main body (621); and
wherein the first pressure sensor (624) and the second pressure sensor (625) are connected to the control module; and the first pressure sensor (624) and the second pressure sensor (625) are configured to sense the gas pressures subjected to the ventilation side of the diaphragm assembly A (620) and the control side of the diaphragm assembly A (620) respectively, thereby to determine whether the diaphragm main body (621) is functioning properly.

16. The artificial resuscitator device according to claim 12, wherein the gas control valve (6) comprises: a valve seat (630), a valve cover (660), a diaphragm assembly B (650), and a pressure tube (670);
wherein the valve seat (630) is configured to connect the first pipe section (111) to the second pipe section (112) and defines a valve cavity therein; the valve cavity is separated into two communicating cavities (640) by a partition plate (680) disposed in the valve cavity; and one of the two communicating cavities (640) is communicated with the second pipe section (112) and another one of the two communicating cavities (640) is communicated with the first pipe section (111);
wherein the valve cover (660) is disposed on a side of the valve seat (630), the diaphragm assembly B (650) is disposed in the valve cover (660), and the diaphragm assembly B (650) is configured to selectively seal the communicating cavity (640) communicated with the first pipe section (111) of the two communicating cavities (640); and a side of the diaphragm assembly B (650) facing towards the pressure tube (670) is a control side of the diaphragm assembly B (650), a side of the diaphragm assembly B (650) facing towards the two communicating cavities (640) is a ventilation side of the diaphragm assembly B (650), and an area under gas pressure of the control side of the diaphragm assembly B (650) is greater than an area under gas pressure of the ventilation side of the diaphragm assembly B (650);
wherein the pressure tube (670) is disposed in a side of the valve cover (660) facing away from the valve seat (630); and an end of the pressure tube (670) facing away from the valve cover (660) is communicated with a connecting tube (12) branched off the exhaust pipe (11); and
wherein the three-way valve (9) is disposed in a junction between the connecting tube (12) and the pressure tube (670), a pressure relief opening (10) is defined on the connecting tube (12) and is configured to selectively communicate with the pressure tube (670) through the three-way valve (9).

17. The artificial resuscitator device according to claim 10, wherein the control module is configured to compare the current ventilation volume of the respirator (5) obtained according to the gas flow detected by the flow sensor (7) with a set ventilation volume, to determine the current ventilation volume of the respirator (5) equal to the set ventilation volume, and then to switch the three-way valve (9) to a locked gas path to communicate the exhaust pipe (11) with the connecting tube (12), thereby exerting a force on the control side of the gas control valve (6) to close the exhaust pipe (11); and
wherein the control module is configured to determine the current ventilation volume of the respirator (5) less than the set ventilation volume, then to switch the three-way valve (9) to a pressure relief gas path to communicate the connecting tube (12) with the pressure relief opening (10), thereby releasing the force on the control side of the gas control valve (6) to open the exhaust pipe (11).

18. The artificial resuscitator device according to claim 8, further comprising: a frequency dynamic indication module; and
wherein the frequency dynamic indication module is configured to prompt an operator to perform a ventilation operation based on a set breathing time interval of a set frequency, control the three-way valve (9) to switch to the pressure relief gas path through the control module, and control the three-way valve (9) to switch to the locked gas path through the control module after that the ventilation operation ends and a next ventilation time node does not reach.

19. An artificial resuscitator device, comprising:
a respirator (5), comprising:
an inlet one-way valve (2);
an outlet one-way valve (4); and
a gas storage bag (3), disposed between the inlet one-way valve (2) and the outlet one-way valve (4);
an exhaust pipe (11), connected with the outlet one-way valve (4);
a gas control valve (6), disposed on the exhaust pipe (11), and located at a side of the outlet one-way valve (4) facing away from the gas storage bag (3);
a flow sensor (7), disposed on the exhaust pipe (11), and located at a side of the gas control valve (6) facing away from the outlet one-way valve (4);
a gas guiding section (8), connected with the exhaust pipe (11), and located at a side of the flow sensor (7) facing away from the gas control valve (6);
a connecting tube (12), connected with the exhaust pipe (11) through the gas control valve (6);
a three-way valve (9), connected between the connecting tube (12) and a pressure relief opening (10); and
a control module, configured to obtain a current ventilation volume of the respirator (5) based on a gas flow detected by the flow sensor (7) and control whether the three-way valve (9) switches a gas path according to the current ventilation volume to open or close the exhaust pipe (11);
wherein the exhaust pipe (11) comprises: a first pipe section (111) connected to the gas storage bag (3) and a second pipe section (112) connected to the gas guiding section (8); the gas control valve (6) is disposed at a junction of the first pipe section (111) and the second pipe section (112), and the connecting tube (12) is connected with the first pipe section (111), and the flow sensor (7) is disposed on the second pipe section (112).

20. The artificial resuscitator device according to claim 19, wherein an end of the first pipe section (111) communicating with the second pipe section (112) is located inside the second pipe section (112) to form an annular connecting section (113), and the gas control valve (6) is set at an end of the second pipe section (112) near the annular connecting section (113); and
wherein the gas control valve (6) comprises a pressure chamber body (610) connected between the second pipe section (112) and the connecting tube (12), the three-way valve (9) is set at a connection between the connecting tube (12) and the pressure chamber body (610), and the pressure relief opening (10) selectively communicates with the pressure chamber body (610) through the three-way valve (9); a diaphragm assembly A (620) is set at a connection between the second pipe section (112) and the pressure chamber body (610), and the diaphragm assembly A (620) is capable of selectively sealing the end of the first pipe section (111) located inside the second pipe section (112); a side of the diaphragm assembly A (620) facing towards the pressure chamber body (610) is a control side of the diaphragm assembly A (620), a side of the diaphragm assembly A (620) facing towards the annular connecting section (113) is a ventilation side of the diaphragm assembly A (620), and an area under gas pressure of the control side of the diaphragm assembly A (620) is greater than an area under gas pressure of the ventilation side of the diaphragm assembly A (620).
